(19) United States
(12) Patent Application Publication
(10) Pub. No.: US 2006/0122520 A1
(43) Pub. Date: Jun. 8, 2006

(54) VITAL SIGN-MONITORING SYSTEM WITH MULTIPLE OPTICAL MODULES

(75) Inventors: Matthew John Banet, Del Mar, CA (US); Brett George Morris, San Diego, CA (US); Henk Visser, San Diego, CA (US)

Correspondence Address: Triage Wireless, Inc.;Matthew John Banet, 6540 LUSK BLVD., C200, SAN DIEGO, CA, 92121, US

(73) Assignee: Dr. Matthew John Banet, Del Mar, CA (US)

(21) Appl. No.: 10/904,968

(22) Filed: Dec. 7, 2004

(51) Int. Cl. A61B 5/02; A61B 5/00

(52) U.S. Cl. 600/503; 600/323; 600/485; 600/500

(57) ABSTRACT

The invention features a medical device that measures vital signs (e.g., blood pressure, pulse oximetry, and heart rate) from a patient using at least two optical modules. Each optical module typically features two light sources (red, infrared) and a photodetector. Both optical modules are configured to measure time-dependent signals describing the patient's flowing blood. A processor analyzes the time-dependent signals to determine the patient's vital signs. Once the vital signs are measured, a wireless transmitter in the body-worn device transmits them to an external device. Processing signals from least two optical modules compensates for motion-related artifacts and noise normally present in signals used to determine vital signs from a device featuring just a single optical module.

### CROSS REFERENCES TO RELATED APPLICATION

Not Applicable

### STATEMENT REGARDING FEDERALLY SPONSORED RESEARCH OR DEVELOPMENT

Not Applicable

### BACKGROUND OF THE INVENTION

1. Field of the Invention

The present invention relates to medical devices for monitoring pulse oximetry and blood pressure.

2. Description of the Related Art

Pulse oximeters are medical devices featuring an optical module, typically worn on a patient's finger or ear lobe, and a processing module that analyzes data generated by the optical module. The optical module typically features first and second light sources (e.g., light-emitting diodes, or LEDs) that transmit optical radiation at, respectively, red (λ˜630 nm) and infrared (λ˜900 nm) wavelengths. The optical module also features a photodetector that detects radiation transmitted or reflected by an underlying artery. Typically the red and infrared LEDs sequentially emit radiation that is partially absorbed by flowing blood in the artery. The photodetector detects transmitted or reflected radiation and in response generates a separate radiation-induced signal for each wavelength. The signal, called a plethysmograph, varies in a time-dependent manner as each heartbeat varies the volume of arterial blood and hence the amount of transmitted or reflected radiation. A microprocessor in the pulse oximeter processes the relative absorption of red and infrared radiation to determine the oxygen saturation in the patient's blood. A number between 94%-100% is considered normal. In addition, the microprocessor analyzes time-dependent features in the plethysmograph to determine the patient's heart rate.

Pulse oximeters work best when the appendage they attach to (e.g., a finger) is at rest. If the finger is moving, for example, the light source and photodetector within the optical module typically move relative to the hand. This generates ‘noise’ in the plethysmograph, which in turn can lead to motion-related artifacts in data describing pulse oximetry and heart rate. Various methods have been disclosed for using pulse oximeters to obtain arterial blood pressure values for a patient. One such method is disclosed in U.S. Pat. No. 5,140,990 to Jones et al., for a ‘Method Of Measuring Blood Pressure With a Photoplethysmograph’. The '990 patent discloses using a pulse oximeter with a calibrated auxiliary blood pressure to generate a constant that is specific to a patient's blood pressure. Another method for using a pulse oximeter to measure blood pressure is disclosed in U.S. Pat. No. 6,616,613 to Goodman for a ‘Physiological Signal Monitoring System’. The '613 Patent discloses processing a pulse oximetry signal in combination with information from a calibrating device to determine a patient's blood pressure.

### BRIEF SUMMARY OF THE INVENTION

The present invention measures vital signs (e.g., blood pressure, pulse oximetry, and heart rate) from a patient using a body-worn device that features at least two optical modules. Each optical module typically features two light sources (red, infrared) and a photodetector. Both optical modules are configured to measure time-dependent signals describing the patient's flowing blood. A processor analyzes the time-dependent signals to determine the patient's vital signs. Once the vital signs are measured, a wireless transmitter in the body-worn device transmits them to an external device. Processing signals from least two optical modules compensates for motion-related artifacts and noise normally present in signals used to determine vital signs from a device featuring just a single optical module.

In one aspect, the invention features a medical device for measuring vital signs from a patient that includes: 1) a first optical module that includes a first light source and a first photodetector, the first light source and first photodetector oriented to optically measure blood flowing in an underlying artery; 2) a second optical module that includes a second light source and a second photodetector, the second light source and second photodetector oriented to optically measure blood flowing in an underlying artery; and 3) a processor, in electrical communication with the first and second photodetector, configured to run a firmware algorithm that processes signals from the first and second photodetectors to determine at least one vital sign from the patient.

In one embodiment, the first and second optical modules are included in a finger-worn component, e.g. a ring, or a component that attaches to the patient's ear or forehead. Alternatively, the first and second optical modules operate in a ‘reflection mode’ geometry and can be attached to any part of the patient's body that includes an underlying artery. In another embodiment, the firmware algorithm running on the processor calculates the patient's pulse oximetry, heart rate, and blood pressure by first averaging signals from the first and second optical modules. Alternatively, the firmware algorithm selects a preferred signal from at least one of the modules, e.g. a signal that has an optimal signal-to-noise ratio.

In another embodiment, the medical device additionally includes a short-range wireless component that sends information describing the patient's vital signs to an external device, e.g. a cellular telephone or a personal digital assistant.

Another aspect of the present invention is a pulse oximetry device including an annular body containing at least four light sources, at least four photodetectors, and a pulse oximetry circuit. The annular body has a diameter preferably ranging from 0.5 inch to 3.0 inches. The annular body has an aperture with a diameter preferably ranging 0.40 inch to 2.0 inches. The annular body has a length preferably ranging from 0.10 inch to 2.0 inches. Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is a front view of an optical ring module featuring multiple optical modules for measuring vital signs according to the present invention;

FIG. 2 is a cross-sectional view of the optical ring module and multiple optical modules of FIG. 1;

FIG. 3A is a cross-sectional view of the optical ring module of FIG. 2 surrounding a patient's finger;

FIG. 3B is a cross-sectional view of the optical ring module of FIG. 3A rotated by a few degrees relative to the patient's finger;

FIG. 4 is a schematic view of a microprocessor in electrical communication with the optical modules of FIG. 1;

FIG. 5 is a schematic view of an algorithm for processing the plethysmographs of FIG. 5 to generate a compiled and averaged plethysmograph; and

FIG. 6 shows a semi-schematic view of a system for measuring blood pressure based on the optical ring module of FIG. 1.

### DETAILED DESCRIPTION OF THE INVENTION

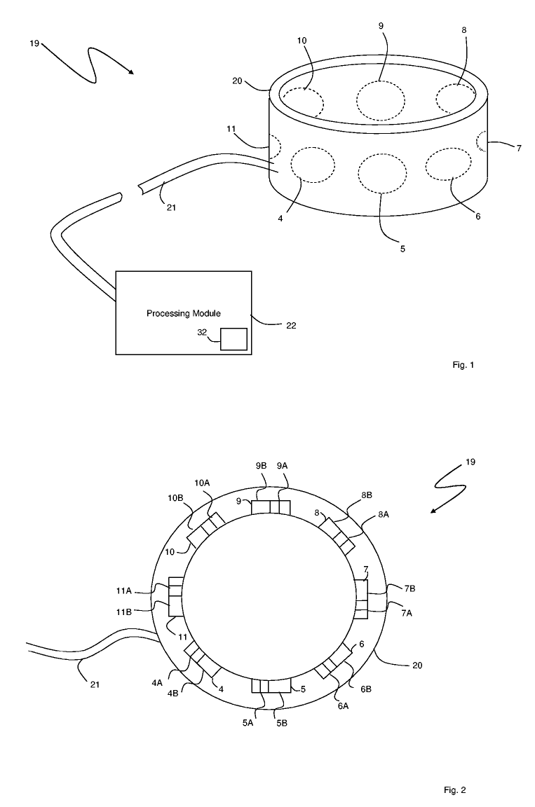

FIGS. 1 and 2 show a medical device 19 according to the invention that features an annular optical ring module 20 that includes multiple optical modules 4-11, each of which measures a plethysmograph from a patient. The optical modules 4-11 are evenly disposed around a perimeter of the ring module 20 and each feature a photodetector 4B-11B that detects radiation, and a pair of LEDs 4A-11A that generate red and infrared radiation. An electrical cable 21 connects the optical modules 4-11 to a processing module 22. When a patient wears the ring module 20 on a finger, each optical module 4-11 simultaneously measures a signal describing the flow of blood in an underlying artery. The signal from each optical module 4-11 passes through the cable 21 to the processing module 22, which includes a microprocessor 32 that processes the signals to determine an individual plethysmograph for each optical module 4-11. An algorithm running on the microprocessor 32 then analyzes the plethysmographs as described below to determine the patient's vital signs (e.g., heart rate, pulse oximetry, and blood pressure).

Multiple optical modules 4-11 within the ring module 20 correct for motion-related artifacts normally present during conventional pulse-oximetry measurements. In one embodiment, for example, the LEDs 4A-11A within each optical module simultaneously emit red, and then infrared, radiation. Radiation from the LEDs 4A-11A forms a symmetrical ‘optical field’ that surrounds the finger and is partially absorbed by pulsing blood in the underlying arteries. Each photodetector 4B-11B detects a portion of the optical field and sends it to the processing module 22 for analysis by a firmware program. In this way, the photodetectors 4B-11B generate an average signal that is relatively independent on the finger's position. Compared to signals from conventional pulse oximeters, the average signal is relatively immune from motion-related artifacts. In another embodiment, LEDs 4A-11A within each optical module sequentially emit radiation in a strobe-like manner. In this case, each photodiode 4B-11B sequentially detects a signal that the processing module 22 analyzes as described above. The processing module 22 runs a firmware program that selects the plethysmograph that is least affected by motion-related artifacts and consequently has the best signal-to-noise ratio. In general, a variety of methodologies for powering the optical modules, coupled with different signal-processing techniques, can be used to analyze plethysmographs generated with the multiple optical modules 4-11 within the ring module 20.

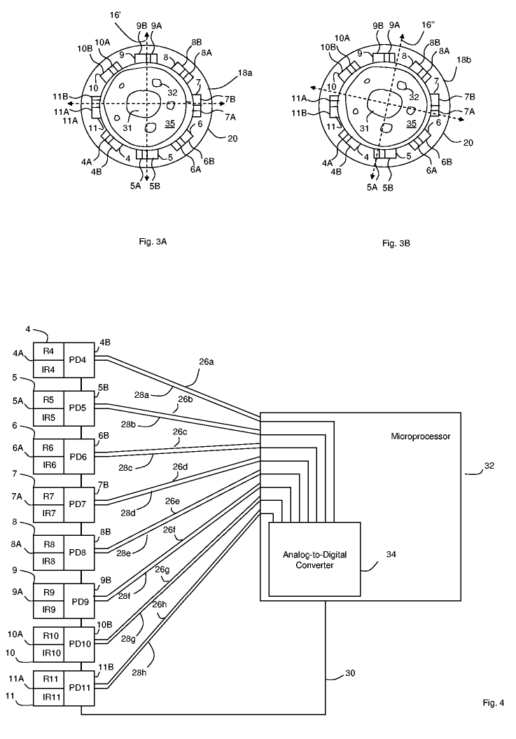

FIGS. 3A and 3B show in more detail how the ring module 20 featuring multiple optical modules 4-11 effectively compensates for motion-related artifacts. Referring first to FIG. 3A, the ring module 20 surrounds a patient's finger 35 that includes several arteries 32 and a bone 31. A first axis 16′ describes the relative position of the finger 35 to the ring module 20. During a measurement, the LEDs 4A-11A can either emit radiation simultaneously or sequentially as described above. The radiation scatters off the bone 31 and tissue in the finger 35 to form a constant, symmetric optical field that surrounds the underlying arteries 32. The photodetectors 4B-11B collect both reflected and transmitted portions of the optical field to generate a collection of radiation-induced signals that a microprocessor then analyzes to determine an average plethysmograph. Because of the configuration of the optical modules 4-11, the optical field is constant regardless of how the finger 35 and arteries 32 are oriented. For example, in FIG. 3B a second axis 16″ shows how movement in the patient's hand rotates the finger 35, bone 31, and the underlying arteries 32 a few degrees relative to the multiple optical modules 4-11. Since the optical modules 4-11 surround the finger 35, however, the LEDs 4A-11A still radiate the arteries 32 with an optical field that is the same as that for FIG. 3A. This means the resultant plethysmograph is basically independent of the relative position between the ring module 20 and the patient's finger 35 and is consequently immune to motion.

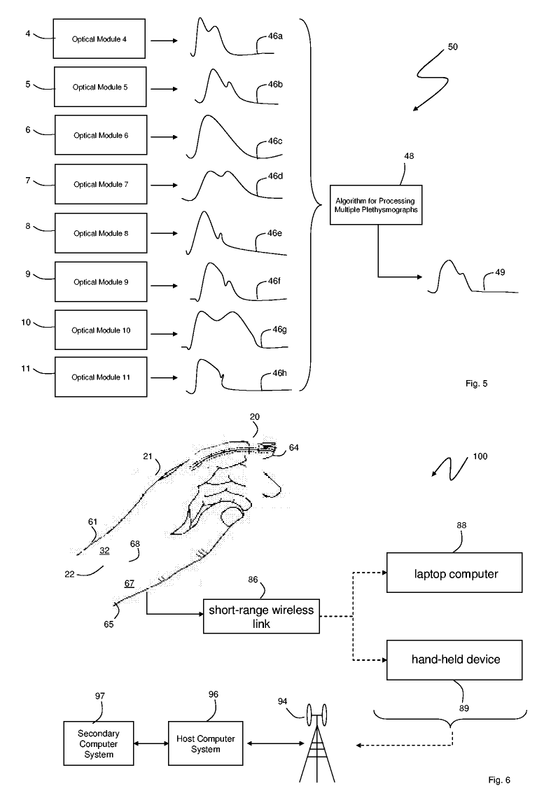

FIG. 4 shows in detail how the microprocessor 32 within the processing module 22 of FIG. 1 collects and processes signals from each optical module 4-11 in the ring module 20. The microprocessor 32 features an analog-to-digital converter 34 that includes multiple channels that each connect through a first electrical lead 28a-h to the individual optical modules 4-11. Each channel converts an analog signal from an optical module into a digital signal that can be processed as described below to determine the patient's vital signs. The microprocessor also includes a second electrical lead 26a-h that supplies power to the LEDs 4A-11A and photodetectors 4B-11B in each optical module. A third electrical lead 30 connects to the microprocessor 32 and each optical module 4-11 to provide a ground for powering the LEDs 4A-11A and photodetectors 4B-11B, as well as a ground for the signal transported by the first electrical lead 28a-h. During operation, the microprocessor 32 supplies power and ground to each optical module 4-11 through, respectively, the second 26a-h and third electrical lead 30. In response to reflected and/or transmitted optical radiation, each optical module 4-11 generates photocurrent that passes as an analog signal through the second electrical lead 28a-h to the analog-to-digital converter 34. The analog-to-digital converter 34 converts the analog signal to a digital signal, which the microprocessor 32 then processes to determine a plethysmograph. The microprocessor 32 additionally runs a firmware program that controls the LEDs 4A-11A and photodetectors 4B-11B in each optical module 4-11. The firmware program, for example, may power each optical module 4-11 simultaneously or sequentially as described above with reference to FIGS. 1-3.

FIG. 5 shows a process 50 for measuring and processing multiple plethysmographs 46a-46h from the optical modules 4-11 with an algorithm 48 to generate an ‘optimal’ plethysmograph 49. During the process 50 the optical modules 4-11 are powered either simultaneously or sequentially as described above to generate analog signals that the analog-to-digital converter converts to digital plethysmographs 46a-h. The algorithm 48 receives the digital plethysmographs 46a-h and processes them to determine the optimal plethysmograph 49. In one example, the algorithm 48 averages all the plethysmographs 46a-h to determine the optimal plethysmograph 49. Or it may select the plethysmograph with the best signal-to-noise ratio, or that which can be best represented by a mathematical model. In still other embodiments, the microprocessor takes a Fourier transform of each plethysmograph 46a-h, and then processes the transforms to generate the optimal plethysmograph 49.

The optimal plethysmograph 49, once generated, can be processed to determine vital signs such as heart rate, pulse oximetry, and blood pressure. Methods for determining heart rate and pulse oximetry from the plethysmograph are well known and are briefly described above. Methods for determining systolic and diastolic blood pressure from the plethysmograph typically involve calibrating a device with a conventional blood pressure monitor to correlate features of the plethysmograph to blood pressure. Specific methods for processing the plethysmograph to determine blood pressure are described in the following co-pending patent applications, the entire contents of which are incorporated by reference: 1) U.S. patent Application Ser. No. 10/967,610, filed Oct. 18, 2004, for a BLOOD PRESSURE MONITORING DEVICE FEATURING A CALIBRATION-BASED ANALYSIS; 2) U.S. patent application Ser. No. 10/810,237, filed Mar. 26, 2004, for a CUFFLESS BLOOD PRESSURE MONITOR AND ACCOMPANYING WEB SERVICES INTERFACE; 3) U.S. patent application Ser. No. 10/709,015, filed Apr. 7, 2004, for a CUFFLESS BLOOD-PRESSURE MONITOR AND ACCOMPANYING WIRELESS, INTERNET-BASED SYSTEM; and 4) U.S. patent application Ser. No. 10/752,198, filed Jan. 6, 2004, for a WIRELESS, INTERNET-BASED MEDICAL DIAGNOSTIC SYSTEM.

FIG. 6 shows a monitoring system 100 that measures a patient's vital signs using the above-described ring module 20 and processing module 22. The system 100 features a wrist-worn monitoring device 68 that measures vital signs as described above and wirelessly transmits them through a short-range wireless link 86 to an external laptop computer 88 or hand-held device 89. The monitoring device 68 preferably includes a wrist-mounted module 61 that attaches to an area of the user's wrist 65 where a watch is typically worn. The ring module 20 typically attaches to the patient's index finger 64. An electrical cable 21 provides an electrical connection between the ring module 20 and wrist-mounted module 61. Preferably the wrist-mounted module 61 includes a microprocessor 32 and a short-range wireless transceiver 67. The components are typically embedded within a comfortable, non-conductive material, such as neoprene rubber, that wraps around the patient's wrist.

The short-range wireless transceiver 67 is preferably a transmitter operating on a wireless protocol, e.g. Bluetooth™, 802.15.4 or 802.11. During operation, the short-range wireless transceiver 67 receives information from the microprocessor 32 and transmits this in the form of a packet to the external laptop computer 88 or hand-held device 89. In certain embodiments, the hand-held device 89 is a cellular telephone with a Bluetooth™ circuit and antenna integrated directly into a chipset used therein. In this case, the cellular telephone may include a software application that receives, processes, and displays the information. Both the hand-held device 89 and laptop computer 88 may also include a long-range wireless transmitter that transmits information over a network 94, e.g. a terrestrial, satellite, or 802.11-based wireless network. Suitable networks include those operating at least one of the following protocols: CDMA, GSM, GPRS, Mobitex, DataTac, iDEN, and analogs and derivatives thereof. In this case, the network 94 connects to an Internet-based host computer system 96 that can display the patient's vital signs on a website. A user then accesses this information using a secondary computer system 97. A detailed description of this component of the invention can be found in the above-mentioned patent applications, previously incorporated by reference, and in U.S. patent application Ser. No. 10/709,015, filed Apr. 7, 2004, for a CUFFLESS BLOOD-PRESSURE MONITOR AND ACCOMPANYING WIRELESS MOBILE DEVICE, the contents of which are also incorporated herein by reference.

In other embodiments, the above-described device for measuring vital signs can include between about one and twenty optical modules. These optical modules are typically included in a finger or wrist-worn device, but alternatively can be included in a device that attaches to a patient's ear or forehead. Typically the optical modules are disposed in a symmetric configuration. Alternatively, the modules can be disposed in a non-symmetric configuration, i.e. they can be grouped in a particular area on the device. In this case the processing module may be worn on the patient's body, e.g., on the patient's waist. Or the optical modules can operate in a ‘reflection mode’ geometry and attach to any part of the patient's body that includes an accessible artery.

The microprocessor can implement a wide variety of algorithms to compensate for motion and calculate vital signs from the patient. For example, the microprocessor may use a Fourier Transform algorithm to determine an optimal time to collect plethysmographs from the multiple optical modules.

Still other embodiments are within the scope of the following claims.

What is claimed is:

1. A medical device for measuring vital signs from a patient, comprising: a first optical module comprising a first light source and a first photodetector, the first light source and first photodetector oriented to optically measure blood flowing in an underlying artery; a second optical module comprising a second light source and a second photodetector, the second light source and second photodetector oriented to optically measure blood flowing in an underlying artery; and a processor, in electrical communication with the first and second photodetector, configured to run a firmware algorithm that processes signals from the first and second photodetectors to determine at least one vital sign from the patient.

2. The medical device of claim 1, wherein the first and second optical modules are comprised by a finger-worn component.

3. The medical device of claim 2, wherein the first and second optical modules are comprised by a ring configured to be worn on the patient's finger.

4. The medical device of claim 1, wherein the first and second optical modules are comprised by a component that attaches to the patient's ear or forehead.

5. The medical device of claim 1, wherein the processor comprises a microprocessor.

6. The medical device of claim 5, wherein the microprocessor comprises an analog-to-digital converter that receives analog signals from the first and second photodetectors and converts them into digital signals.

7. The medical device of claim 6, wherein the firmware algorithm processes the digital signals to determine at least one vital sign.

8. The medical device of claim 1, wherein the firmware algorithm is configured to process the signals from the first and second photodetectors to at least determine the patient's pulse oximetry, heart rate, and blood pressure.

9. The medical device of claim 1, further comprising a short-range wireless component that sends information describing the patient's vital signs to an external device.

10. The medical device of claim 1, further comprising a wrist-worn component.

11. The medical device of claim 10, wherein the first and second optical modules and the processor are comprised by the wrist-worn component.

12. The medical device of claim 1, wherein the firmware algorithm is configured to average signals from at least the first and second optical modules.

13. The medical device of claim 1, wherein the firmware algorithm is configured to select at least one signal from at least the first and second optical modules.

14. A medical device for measuring blood pressure from a patient, comprising: a first optical module comprising a first light source and a first photodetector, the first light source and first photodetector oriented to optically measure blood flowing in an underlying artery; a second optical module comprising a second light source and a second photodetector, the second light source and second photodetector oriented to optically measure blood flowing in an underlying artery; and a processor, in electrical communication with the first and second photodetector, configured to run a firmware algorithm that processes signals from the first and second photodetectors to determine a blood pressure value from the patient.

15. The medical device of claim 15, wherein the first and second optical modules are comprised by a finger-worn component.

16. The medical device of claim 15, wherein the first and second optical modules are comprised by a component that attaches to the patient's ear or forehead.

17. A medical device for measuring vital signs from a patient, comprising: a first optical module comprising a first light source and a first photodetector, the first light source and first photodetector oriented to optically measure blood flowing in an underlying artery; a second optical module comprising a second light source and a second photodetector, the second light source and second photodetector oriented to optically measure blood flowing in an underlying artery; a processor, in electrical communication with the first and second photodetector, configured to run a firmware algorithm that processes signals from the first and second photodetectors to determine at least one vital sign from the patient; and a short-range wireless component, in electrical communication with the processor, configured to send vital sign information to an external device.
